(19) United States
(12) Patent Application Publication
(10) Pub. No.: US 2023/0309512 A1
(43) Pub. Date: Oct. 5, 2023

(54) Adjustable Gaiter, Especially for Horses

(71) Applicant: Carsten Engelke, Lindwedel (DE)

(72) Inventor: Carsten Engelke, Lindwedel (DE)

(21) Appl. No.: 18/170,245

(22) Filed: Feb. 16, 2023

(30) Foreign Application Priority Data
Feb. 18, 2022 (DE) 102022201733.1

(51) Int. Cl. A01K 13/00

(52) U.S. Cl. CPC A01K 13/007; B68C 5/00

(57) ABSTRACT

A gaiter which has as an insert an elastic sheath which has at least one projecting region as a retaining projection, one curved shell encompassing the insert and having two opposite legs, the shell having retaining recesses arranged to match the retaining projections of the insert, which retaining recesses have a larger cross-section than the retaining projections of the insert.

### PRIORITY CLAIM AND REFERENCE TO RELATED APPLICATION

This application claims priority of the German patent application 10 2022 201 733.1, application date 18 Feb. 2022.

### FIELD OF THE INVENTION

The present invention relates to a gaiter for use as a support for a joint, preferably for animals, in particular for horses, wherein the gaiter is to be arranged around an ankle or hock to support the joint.

### BACKGROUND

DE 40 29 120 A1 describes a gaiter which, in order to adapt to a gaskin and foot, has sleeves which are filled with fillers and can be vacuumed, which sleeves completely encompass the gaskin and which are firmly enclosed by shell parts.

US 2019/0021893 A1 describes an orthopedic support for a joint in which internal cushions are firmly inserted into holes in rigid frame members by means of protruding mushroom heads, with an elastic interlayer between the pad and the frame member such that the shaft of the mushroom head protrudes through the elastic interlayer and the mushroom head protrudes into the recess of the frame member.

### SUMMARY OF THE INVENTION

A gaiter of the invention provides a precise fitting form when placed on the joint and e.g. adapts and adjoins to the surface of the leg. The gaiter also forms a stable and firm support and provides external protection against mechanical impacts. The gaiter has the advantage that it allows for movements of an enclosed joint and preferably adapts to such movements.

The gaiter is suitable for horses or other animals and also suitable for use on humans, especially in the area of joints, for example, at the ankle or wrist.

### BRIEF DESCRIPTION OF THE DRAWINGS

The invention is now described with reference to the figures, which schematically show the gaiter in

FIGS. 1A and 1B in exploded view of a sleeve and a shell,

FIG. 2 as a sleeve arranged in the shell,

FIGS. 3A to 3C as individual and assembled parts in one embodiment,

FIGS. 4A to 4C as individual and assembled parts in a further embodiment,

FIGS. 5A to 5E as individual and assembled parts in a further embodiment,

FIGS. 6A to 6E as individual and assembled parts in a still further embodiment,

FIG. 7 an embodiment of a retaining recess with spring elements connected to a retaining receptacle, and

FIG. 8 schematically an insert divided into insert segments.

In the figures, identical reference numerals indicate functionally identical parts.

### DETAILED DESCRIPTION OF THE PREFERRED EMBODIMENTS

The invention has the object to provide a gaiter which can be easily fitted around an ankle or hock of an animal, in particular of a horse, which gaiter on the one hand fits matchingly over substantially its entire surface and on the other hand forms a stable support without stiffening the joint and forms a protection against an external impact. Preferably, the gaiter shall form a support for a region of a joint, which support acts in the event of sudden loading of the joint, but is moldable in the absence of sudden loading. Preferably, the gaiter shall be easy to put on and easy to clean.

The invention achieves the object by the features of the claims and in particular provides a gaiter which comprises or consists of,
as an insert, an elastic sleeve containing a moldable mass and optionally having a valve, wherein the insert has at least one projecting region as a retaining projection, preferably at least one projecting region as a retaining projection on each of at least two opposite regions of its outer wall,
a curved shell encompassing the insert and having two opposing legs, the curved shell having retaining recesses which are arranged to match the retaining projections of the insert and which have a larger cross-section than the retaining projections of the insert in order to form a floating retainer of retaining projections in one retaining recess each,
with at least one tab which is releasably attachable to opposing legs of the shell to bridge the clearance between the opposing edges of the legs and to tension the opposing legs toward each other,
wherein optionally a shell segment movable relative to the shell is arranged in the shell, in particular terminally, which shell segment has at least one tab which can be detachably attached to opposite legs of the shell segment,
further optionally an intermediate layer, e.g. single-layer elastic, optionally foamed plastic, in particular single-layer foamed neoprene, arranged in the shell.

Optionally, the insert has exactly two retaining projections, which are further preferably arranged at a first terminal section of the insert, which terminal section extends over at most 30%, preferably at most 25% or at most 20% or at most 10% to 15% of the length of the insert along its longitudinal extent and along the longitudinal extent of the shell. The longitudinal extent of the insert lies in parallel to the longitudinal extent of the shell, respectively in parallel to the longitudinal axis of the shell, and in perpendicular to the cross-section of the shell. Optionally, the insert at its first terminal section, which is preferably adapted to be arranged on the cannon bone of a horse, has exactly two retaining projections which engage in retaining recesses matchingly arranged on the first terminal section of the shell, wherein the first terminal section of the shell is adapted to firmly encompass the first terminal section of the insert. Therein, the first terminal section of the shell and of the insert preferably lies at the first end of the shell which has a smaller internal cross-section and/or opposite to the second end of the shell and/or of the insert at which the second terminal section of the shell and optionally the second terminal section of the insert has an internal cross-section larger than the opposite respective first terminal section. The shell preferably has a larger internal cross-section at its second terminal section than at its first terminal section, the second terminal section being provided to be arranged at a distance from the insert around the region of a joint, in particular around the fetlock joint of a horse, while the first end of the shell is provided to be arranged adjacent the first terminal section of the insert, which terminal section is arranged to lie against the cannon bone. The second terminal section of the shell is configured to be movable or to be arranged at a distance from the insert, in particular at a distance from the second terminal section of the insert, so that the second terminal section of the insert can move away from the second terminal section of the shell, for example during a movement in the fetlock joint. Therefore, the second terminal section of the shell is not fixedly connected to the second terminal section of the insert, but the second terminal section of the shell is pivotably hinged against the second terminal section of the insert around the retaining projections engaging in the retaining recesses at the first terminal section of the shell or insert, and is preferably floatingly and displaceably guided.

Due to the internal cross-section at the second terminal section of the shell being larger than the one at the first terminal section, the gaiter according to the invention is configured such that the insert at its first terminal section lies firmly against a cannon bone by means of the first terminal section of the shell, and that the second terminal section of the insert is pivotable into a distance from the second terminal section of the shell during movement in the fetlock joint. The insert can be adapted with its moldable mass to match the cannon bone and/or fetlock joint in a position of the fetlock joint, in particular by solidifying by means of vacuuming a moldable mass formed by loose particles in the sleeve. Therein, the shell covers the insert and the insert at least in the second terminal section is guided pivotably to the shell.

The internal cross-section of the shell at its second terminal section may have a diameter larger than the first terminal section, for example, by 10% to 20% and/or by 2 to 10 cm or up to 8 cm.

The legs of the shell preferably form a U-shaped cross-section of the shell. The shell may comprise or consist of legs symmetrical along a central plane. The sleeve may have a bent rectangular perimeter, preferably have a cross-section having two opposing legs arranged exclusively within the shell, respectively on the inside of the shell and not projecting beyond the shell.

The retaining recesses may be breaches in the shell through which the projecting regions extend that form the retaining projections. Optionally, the retaining recesses are indentations extending from the concave inner surface of the shell, wherein the opposite convex outer surface of the shell has a closed surface which may optionally be bulging to the outside in the area of the recesses.

Preferably, the projecting regions of the sleeve forming the insert and the matchingly aligned retaining recesses of the shell are arranged along the opposing edges of the legs, e.g., at least two each, at least three each, at least four each along each of the two opposing edges.

The moldable mass is generally one that can assume a moldable state and a solid state at ambient temperature and at body temperature, e.g., between 5 and 40° C., wherein the state is adjustable or the mass assumes the state depending on the external load.

The moldable mass can be formed by particles, e.g. by plastic spheres, e.g. made of plastic foam, in particular foamed polystyrene, foamed ceramic or solid or hollow particles, preferably spheres. The particles may be loosely movable relative to each other in a moldable state or may be present movably in a mixture with a dilatant fluid. For example, the particles may have a diameter of 1 to 10 mm, preferably 2 to 7 or up to 5 mm.

The sleeve forming the insert is airtight and, in the embodiment in which the moldable mass is formed by particles, is evacuable through a valve which is preferably accessible through an additional breach in the shell, so that the particles contained in the sleeve form a solid pack as a solid state after vacuuming, while the particles are movable relative to one another when the valve is open and form a loose bulk which conforms to the sleeve. Generally preferably, the valve is not fixedly attached to the shell, but passes, for example, through an opening in the shell that is larger than the valve. Preferably, the valve is displaceably, also referred to as floatingly, held in the shell, for example displaceable along two axes in parallel to the surface of the shell, each displaceable by at least 1 mm, preferably at least 2 mm or at least 3 mm, for example displaceable by up to 15 mm, up to 12 mm, up to 9 mm or up to 6 mm.

The moldable mass may be formed by a dilatant fluid, such as boron-containing polydimethylsiloxane, also known as borosilicone putty, or by a boric acid-containing aqueous solution of polyvinyl alcohol. A dilatant fluid as a moldable mass in at least one segment of the sleeve has the advantage of being deformable under steady load and being adaptable to a joint in this moldable state, and that under sudden load the dilatant fluid becomes more rigid, assumes a solid state, and forms a solid support for an adjacent joint.

The insert is preferably held displaceably, also referred to as floatingly, in the shell, e.g. displaceable along two axes in parallel to the surface of the shell, in each case by at least 1 mm, preferably displaceable by at least 2 mm or at least 3 mm, e.g. by up to 15 mm, up to 12 up to 9 mm or up to 6 mm, preferably held exclusively by its projecting regions, which form retaining projections and which protrude into the matchingly arranged retaining recesses of the shell. The insert is therefore easy to connect to the shell and also easy to disconnect from the shell, e.g. for cleaning of both. By the retaining projections engaging in retaining recesses of larger cross-section, the insert is mounted on the shell in a displaceable or floating manner, in particular preferably in that retaining projections arranged exclusively on the first terminal section of the insert engage in a displaceable manner in retaining recesses arranged on the first terminal section of the shell.

Optionally, the insert, e.g., a segment of the sleeve, may be connected to the shell by a releasable adhesive connection, e.g., a hook-and-loop fastener, e.g., exclusively along or transverse to a central plane, so that the two regions of the insert lying on either side of the adhesive connection are freely movable in the shell and only with their retaining projections protrude into the retaining recesses of the shell.

Generally preferably, at least one, more preferably at least two, of the retaining projections is held in an elastically floating manner in a retaining recess in each case, e.g. by means of spring elements which extend in the retaining recess up to a retaining projection. Therein, at least one spring element may be attached to the shell, preferably two or three spring elements, each of which holds a retaining projection on or in the retaining recess, the retaining projection being set up to latch or clamp a retaining projection. The spring elements may be connected to or form an annular retaining receptacle such that a retaining projection may be clamped into the retaining receptacle. A resilient and floating mounting of each retaining projection in a retaining recess, for example in a retaining receptacle elastically held in the retaining recess, has the advantage that the insert can move within the shell, for example by the distance between the retaining recess and the retaining projection, and the insert is tensioned by the spring action into a rest position of the spring element. Preferably, a retaining projection is arranged centrally in the retaining recess in the rest position of the spring element, in which the insert is not tensioned along the longitudinal extent of the shell. A spring element can be formed, for example, as a spoke, straight, bent or arcuate, e.g. arranged radially or at an angle to the radial between the retaining recess and retaining receptacle, made of plastic or spring steel, which extends between the retaining recess and retaining receptacle.

A shell segment, which generally is optional, is preferably arranged at the second terminal section of the shell. Optionally, the insert and the shell and the optional shell segment have only smooth surfaces, wherein optionally no intermediate material such as a fleece, a gel, or a lubricating compound is arranged between the sleeve and the shell or between the sleeve and the joint. This is because it has shown that when the gaiter is attached to the joint, there is little frictional force between the sleeve and the shell, so that no fabric reducing the frictional effect is required, and the gaiter has the added advantage of being easy to clean. Alternatively, an intermediate fabric, such as fleece or foam, may be disposed between the sleeve and the shell, which sleeve forms the insert.

The insert may be single-pieced and may extend beyond the length of the shell and the optional shell segment so that a section of the insert is encompassed by the shell and a section, particularly a terminal section of the insert is encompassed by the optional shell segment.

Preferably, the insert in perpendicular to its longitudinal extension has a width that extends by 180° to 230° or to 210° or to 180°, e.g. from 180 to 200° within the cross-section spanned by the insert and/or by the shell, so that the insert does not encompass the full circumference of 360° of a leg, in particular of a cannon bone or arm, but only a portion of at most 230°, in particular for use for arranging the insert against the back of a leg, in particular of a cannon bone of a horse, in order to support the tendon running along the leg, for example the superficial and deep flexor tendon of a horse. The shell may extend around the same or a greater portion of the circumference of a leg, preferably by at least 10 to 30° more than the insert, such that the shell completely covers the insert.

Optionally, the sleeve forming the insert is divided into interconnected insert segments, which are produced, for example, by welding together strip-shaped regions of the inner wall and of the outer wall of the sleeve which lie opposite to one another, preferably approximately in parallel to or at an angle of at most 30° or at most 20° to the longitudinal extent of the shell. Therein, the insert segments are joined to one another, for example, in that the welding is not continuous, for example, as a sequence of spaced-apart spot-shaped or line-shaped welds, and/or the welding leaves at least one region between insert segments open, for example adjacent to the circumferential edge.

Optionally, insert segments formed by segments of the sleeve are spaced apart and interconnected by a conduit, e.g., formed by a region of the sleeve, particularly by interruptions in welds, and have only one common valve.

The insert segments formed by segments of the sleeve may be spaced apart, may have separate internal volumes and may each have a separate valve, or may be gas-tight and without a valve if the moldable mass comprises or consists of a dilatant fluid, optionally in admixture with loose particles. Segments of the sleeve which are spaced apart from each other, which each contain particles and which are optionally vacuumable by a separate valve each or which have no valve, may be connected to each other, e.g. attached to a common film section, e.g. by a section of plastic film in which the segments are formed, or the segments may be formed completely separate from each other. Segments of a sleeve each form segments of the insert, such that one segment is encompassed by the shell and another segment of the insert is encompassed by a shell segment. Optionally, the insert may have at least one segment with less moldable mass than adjacent segments or without moldable mass, e.g., a central insert segment or an insert segment arranged asymmetrically with respect to a central longitudinal axis with more moldable mass, with less moldable mass, or without moldable mass, which optionally is attached at its periphery, e.g., on all sides or on both sides to at least one insert segment with different content and/or otherwise to insert segments each having the same content of moldable mass, e.g. in order to form a larger inner cross section in the region of the insert segment with less moldable mass, into which, for example, pressure-sensitive regions of an arm or leg, in particular of an ankle or foot joint, can extend. Therein, the content of moldable mass is the content per cm³ of the maximum volume of the insert segment or the content per cm² of the area of the insert segment that extends in parallel to the shell.

Generally, insert segments may be arranged, adjacent to one another or spaced apart, in a row along the longitudinal axis of the shell. Optionally, a segment of the sleeve encompassed by a terminal region of the shell or by a shell segment that is separated from other segments of the sleeve contains a dilatant fluid, optionally in admixture with particles. A segment of the sleeve containing a dilatant fluid, optionally in admixture with particles, may be airtight and without a valve. A segment of the sleeve filled with dilatant fluid, optionally in admixture with particles, may generally form an insert having at least one retaining projection of its own, which retaining projection projects into a matchingly arranged retaining recess of the shell or of the shell segment, the retaining recess having a cross-section equal to or greater than the cross-section of the retaining projection. Alternatively, a segment of the sleeve containing a dilatant fluid, optionally in admixture with particles, particularly when said segment of the sleeve is not connected to other segments of the sleeve, may be fixed to a terminal portion of the shell or to a shell segment, e.g., by means of an adhesive bond, optionally with an intermediate elastic cushioning layer, e.g., foam, which may be, for example, foamed neoprene.

Optionally, the insert is formed by a vacuumable sleeve, e.g. formed of plastic film, within which is arranged a gas-permeable inner layer, e.g. of fabric, woven or fleece, filled with particles. The inner layer may be subdivided into segments which are subdivided, for example, by interconnected areas of the inner layer, such as darts or weld seams, which are filled with particles, in each case to an equal or different volume fraction of the segments. Preferably, segments of the inner layer or the interconnected areas of the inner layer separating them run in parallel to the longitudinal extent of the insert.

Preferably, the shell encompassing the insert protrudes over the circumferential edge of the insert by at least 1 mm, preferably at least 2 mm or at least 3 mm, e.g. up to 15 mm, up to 12 mm, up to 9 mm or up to 6 mm. Then the gaiter has the advantage that the sleeve can follow a movement of the joint undisturbed even after evacuation and, in particular, does not rub against the joint.

The optional shell segment preferably has an outer wall which is arranged adjacent to, optionally with a spacing, within the shell so that the shell segment is encompassed by the shell and is movable in the shell, in particular is arranged rotatably against a longitudinal axis or about a transverse axis of the shell. The shell segment may have retaining projections projecting, for example, approximately in perpendicular to the longitudinal axis of the shell beyond the outer wall of the shell segment, wherein these retaining projections engage matchingly arranged recesses of the shell. Preferably, the recesses of the shell have a larger cross-section than the retaining projections of the shell segment in order to guide the shell segment displaceably along the longitudinal axis of the shell and/or rotatably against the longitudinal axis or about a transverse axis of the shell.

The optional shell segment may be connected to the shell by being connected to the insert and/or to an intermediate layer arranged within the shell.

The shell segment and the tab fixable thereto circumferentially encompass the insert, preferably at one of its terminal sections, so that the shell segment forms an enclosure of a preferably terminal section of the insert which is movable relative to the shell and is rigid in itself.

The optional intermediate layer is preferably arranged between, on the one side, the insert and, on the other side, the shell and a shell segment arranged therein, alternatively, the optional intermediate layer may be arranged between, on the one side, the insert and the shell segment and, on the other side, the shell. Further alternatively, the intermediate layer can be arranged on the surface of the insert that lies opposite to the shell.

Preferably, the intermediate layer has recesses arranged to match the retaining projections of the insert, in which recesses the retaining projections engage. The retaining projections, which engage in the retaining recesses of the shell, hold the insert and the optional intermediate layer at the shell. Alternatively, the intermediate layer may be fixedly connected to the insert or to the shell and/or to the shell segment, e.g. may be attached to the outer side of the insert facing the shell, e.g. by a fixed connection, e.g. adhesive connection or welded connection, or by a detachable connection, e.g. a hook-and-loop fastener.

The gaiter is suitable for use as support for a foot or ankle joint of, for example, a human, preferably of an animal, in particular of a horse, wherein the gaiter is applied to the foot or ankle joint with the valve being open, e.g. with slight pressure until the sleeve forming the insert is in contact, subsequently the sleeve is vacuumed through the valve, whereby the loose bulk of the particles is compacted into a tight pack and the projecting regions move in the recesses of the shell, and thereafter the opposite legs of the shell are tensioned toward each other by means of the at least one tab to lay the shell against the insert, the shell and the optional shell segment being movable relative to the insert or lying firmly against the insert, the shell segment being movably guided on the shell. Preferably, the gaiter has at least two, more preferably at least three, tabs, for example up to six or up to five or four tabs extending across the spacing of the opposing legs of the shell and capable of tensioning them toward each other.

Optionally, one shell each with at least two sleeves forms a system in which the sleeves are interchangeable and each sleeve has a different filling of moldable mass, e.g., of loose particles and/or of dilatant fluid. The filling may vary, for example, according to the type of particles or content of the sleeve of particles and the type and volume of dilatant fluid. In this embodiment, the gaiter has the advantage that a shell is adaptable to an ankle or foot by selecting and arranging one of the sleeves in the shell.

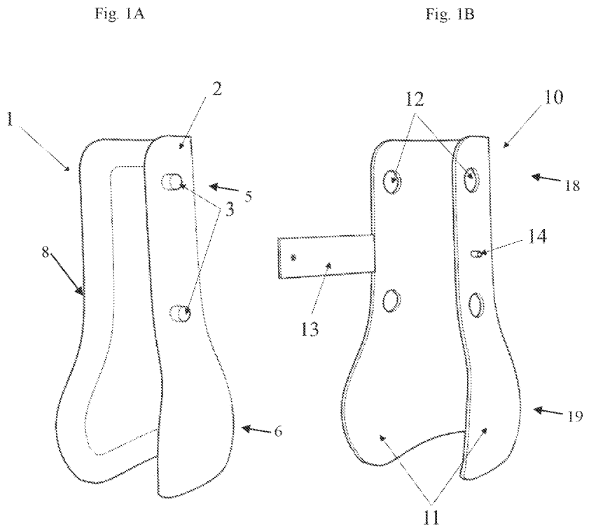

FIGS. 1A-1B shows an insert 1 having two projecting regions 3 on opposite regions of its outer wall 2, respectively, with only the projecting regions forming the retaining projections 3 of the front or right region of the outer wall 2. One of the retaining projections may be a valve 4. Further shown is a curved shell 10 having two opposite legs 11, which has retaining recesses 12 arranged to match the retaining projections 3 of the insert 1, which retaining recesses 12 have a cross-section larger than the projecting retaining projections 3. The insert 1 is bounded by the circumferential edge 8. Representative of at least two tabs, a tab 13 is shown which is releasably attachable to opposite legs 11 of the shell 10 by means of a projection 14 shown here. The first terminal section 5 of the insert 1 lies against the first terminal section 18 of the shell 1, the second terminal section 6 of the insert 1 opposite the first terminal section 5 is arranged in the region of the second terminal section 19 of the shell 1. In embodiments in which the insert 1 has exactly one retaining projection 3 or, preferably, two retaining projections 3 arranged in the first terminal section 5 of the insert 1 in the same region along the longitudinal axis of the shell 10, which is generally preferably equal to the longitudinal axis of the insert 1, which retaining projections 3 are displaceably guided in retaining recesses 12 arranged at the first terminal section 18 of the shell 11, the second terminal section 19 of the shell 1 opposite the first terminal section 18 is pivotable away into a distance from the second terminal section 6 of the insert 1.

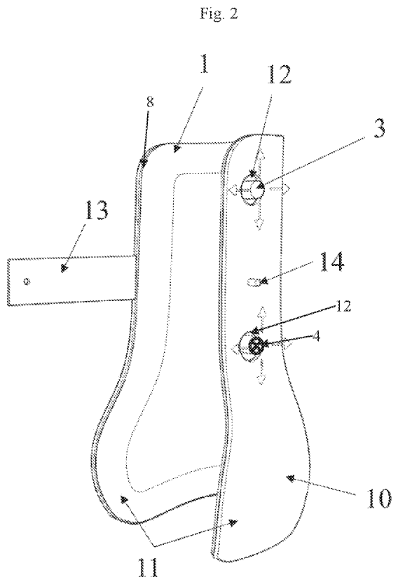

FIG. 2 shows the insert 1 arranged in the shell 10, the projecting retaining projections 3 of which project through retaining recesses 12 shown here as breaches so that the insert 1 is retained in the shell 10 displaceably along the hollow arrows shown here. A valve 4, through which the insert 1 formed as a sleeve can be evacuated or ventilated, can project through a retaining recess 12. The tab 13 can be releasably attached by the projection 14 to bridge the free space lying between the legs 11 of the shell 10 and to tension the opposite legs 11 towards one another.

FIGS. 3-3C show a preferred embodiment in which a one-piece insert 1 (FIG. 1A) formed by a one-piece sleeve is encompassed along its entire length by a shell 10. FIG. 3A shows the shell 10 with retaining recesses 12 arranged in pairs in opposite edge regions, and recesses 15 arranged in a terminal section of the shell 10 to match with retaining projections 21 of a shell segment 20. The shell segment 20, shown separately from the shell 10 in FIG. 3A, with its retaining projections 21 can engage in the recesses 15 of the shell 10 to be pivotally supported against the longitudinal axis 16 of the shell. The shell segment 20 comprises a tab 22 to cover the distance between opposing legs 23 of the shell segment 20 when it is secured to both legs 23.

FIG. 3C shows the gaiter which encompasses an insert 1 with the shell 10, with a shell segment 20 which is movably mounted on the shell 10 between the insert 1 and the shell 10. The movability of the retaining projections 3 of the insert 1 in retaining recesses 12 of the shell 10, and the movability of the retaining projections 21 of the shell segment 20 in recesses 15 of the shell 10 are indicated by the hollow arrows in FIG. 3C. The shell 10 can be closed around the insert 1 by means of the tab 13, and the shell segment 20 can be closed separately around the insert 1 by means of its tab 22.

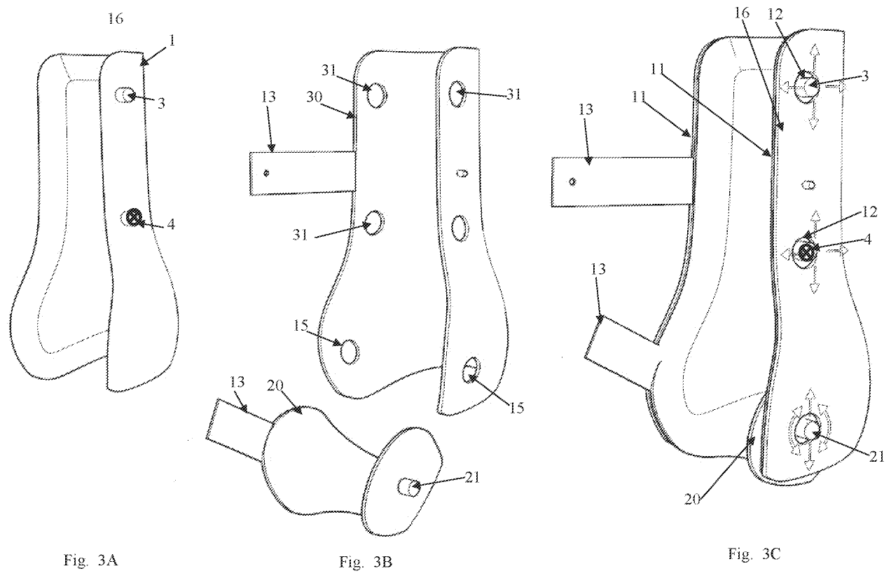

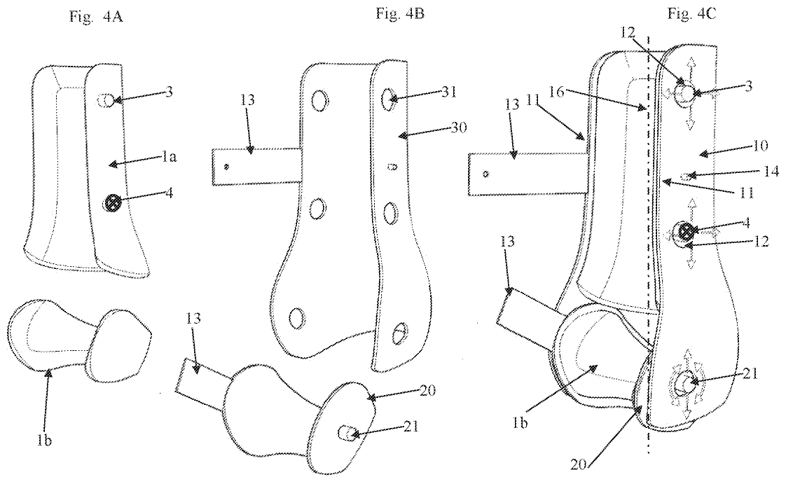

FIGS. 4A to 4C show an embodiment in which the insert 1 is divided into two insert segments 1a, 1b, each of which can be evacuated by means of a separate valve 4. One insert segment 1b is formed to match the inside of a shell segment 12 encompassing the insert segment 1b. The other insert segment 1a is formed to match the area of the inside of the shell 10 that is not covered by the shell segment 20, which is movably arranged in the shell 10. In this embodiment, the insert divided into two insert segments 1a, 1b is movable in accordance with the movement of the shell segment 20 in the shell 10 even when shell segments are evacuated, and is therefore well adapted to the movement of a joint encompassed by the gaiter.

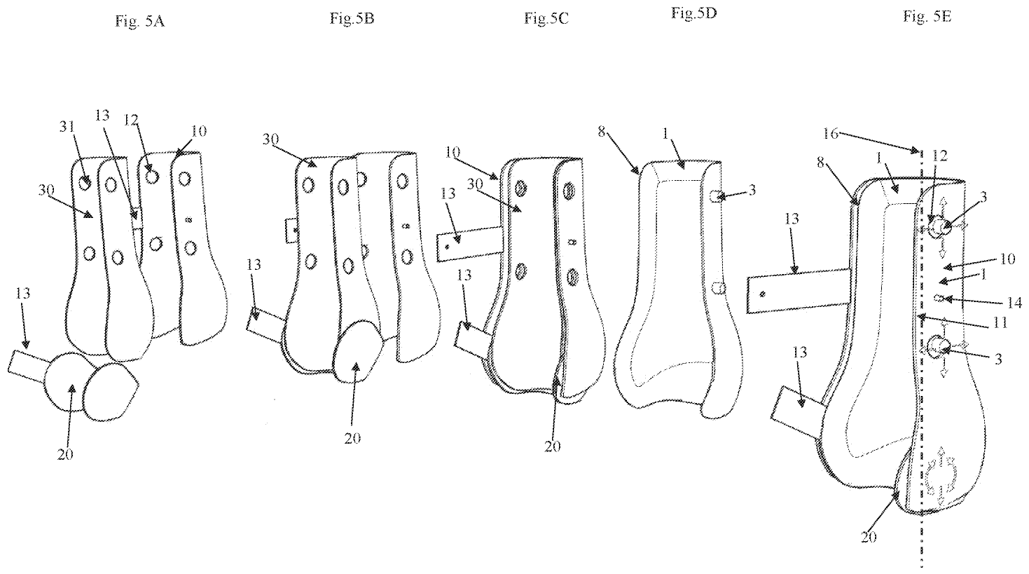

FIGS. 5A to 5E show an embodiment in which an intermediate layer 30 is arranged between, on the one side, an insert 1 and, on the other side, a shell 10 and a shell segment 20. The intermediate layer 30 has recesses 31 arranged to match retaining recesses 12 of the shell 10, so that retaining projections 3 of the insert 1 (FIG. 5D) are arranged through the recesses 31 and the retaining recesses 12 to retain the intermediate layer 30 between the shell 10 and the insert 1 (FIG. 5E). The shell segment 20 is preferably attached to the intermediate layer 30, for example by means of an adhesive or hook-and-loop connection. Alternatively, the shell segment 30 may be loosely arranged between the intermediate layer 30 and the shell 10 and may be loaded or clamped against the intermediate layer 30 with frictional engagement by its tab 22 when the latter encompasses the insert 1.

Also in this embodiment, the shell segment 30 is movably arranged in relation to the shell 10 inside the shell 10 and by the tab 22 encompasses a terminal section of the insert 1.

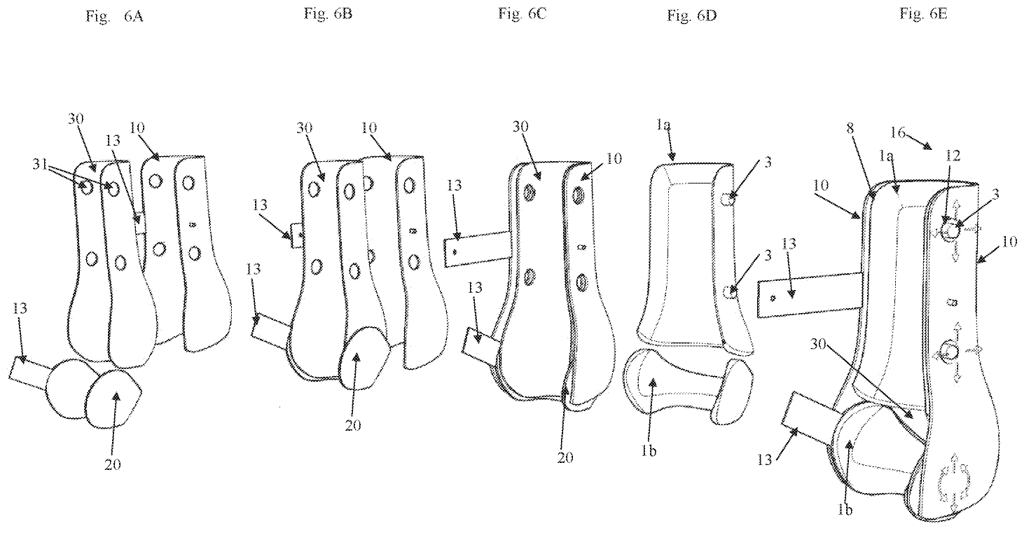

FIGS. 6A to 6E show an embodiment in which the insert 1 is subdivided into insert segments 1a, 1b, of which an insert segment 1b is formed to match the shell segment 20 and is arranged on the latter with an intermediate layer 30 lying between them, wherein preferably the insert segment 1b is attached on one side of the intermediate layer 30 and matchingly covers the shell segment 20 attached on the opposite side of the intermediate layer 30. In this embodiment, it is preferred that the insert segment 1b, which is encompassed by the shell segment 20, contains a dilatant fluid, preferably boron-containing polydimethylsiloxane, as a moldable mass in a sleeve segment.

Also in this embodiment, the shell segment 20 with the insert segment 1b attached thereto is movable relative to the shell 10 due to the flexibility of the intermediate layer 30.

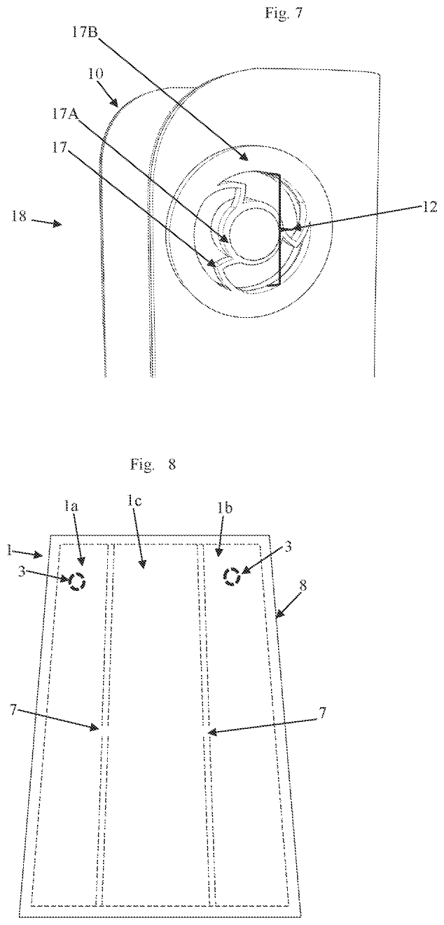

FIG. 7 shows a first terminal section 18 of a shell 1 with a retaining recess 12 in which spring elements 17 extend beyond the cross section of the retaining recess 12 to a retaining receptacle 17A. The retaining receptacle 17A, here annular in shape, has an internal cross-section into which a retaining projection 3 of an insert 1 can be clamped. The spring elements 17, here shown as curved and kinked towards the radial of the retaining recess 12, can consist of plastic or spring steel, for example. Optionally, the spring elements 17 are formed integrally with a spring frame 17B and the retaining recess 17A, for example made of plastic, wherein the spring frame 17B is attached to the shell 10.

FIG. 8 shows an insert 1 divided into insert segments 1a, 1b, 1c which are formed by segments 1a, 1b, 1c of a sleeve and which are spaced apart from one another by an interrupted weld 7, the interrupted weld 7 forming a conduit connecting the insert segments 1a, 1b, 1c to one another. Therefore, the insert segments 1a, 1b, 1c can be vacuumed or pressurized to ambient pressure by a single common valve 4. The circumferential edge 8 of the insert 1 is preferably formed in sections or completely by a weld seam.

### REFERENCE NUMERALS

1 insert
1a, 1b, 1c insert segment
2 outer wall
3 retaining projection
4 valve
5 first terminal section of the insert
6 second terminal section of the insert
7 interrupted weld seam
8 circumferential edge
10 shell
11 leg
12 retaining recess
13 tab
14 projection
15 recess
16 longitudinal axis
17 spring element
17A retaining receptacle
17B spring frame
18 first terminal section of the shell
19 second terminal section of the shell
20 shell segment
21 retaining projection of the shell segment
22 tab of the shell segment
23 leg of the shell segment
30 intermediate layer
31 recesses of the intermediate layer

1. Gaiter comprising an insert encompassed by a shell, wherein the insert is an elastic sleeve containing a moldable mass,
wherein the insert has at least one retaining projection on at least one region of its outer wall,
the shell having two opposite legs which comprise at least one retaining recess arranged to match the retaining projection of the insert and having a cross section larger than the retaining projection,
with at least one tab which can be releasably attached to opposite legs of the shell in order to bridge the free space lying between the legs and to tension the opposite legs towards one another, wherein at least one of the retaining projections is held in a resiliently floating manner in one retaining recess each by at least one spring element which extends in the retaining recess to a retaining projection.

2. Gaiter according to claim 1, wherein the insert has exactly two retaining projections which are arranged at a first terminal section of the insert, which first terminal section extends over a maximum of 30% of the length of the insert along the longitudinal extension of the shell, and the retaining recesses are arranged in a first terminal section of the shell and thereby the second terminal section of the shell is configured to be movable into a distance from the second terminal section of the insert lying opposite to the first terminal section of the insert, wherein the shell in its second terminal section lying opposite to the first terminal section has a larger internal cross-section than in the first terminal section.

3. Gaiter according to claim 1, wherein the insert is divided into at least one insert segment with less moldable mass or without moldable mass and adjacent to at least one insert segment containing more moldable mass, the insert segment with less or without moldable mass and the at least one adjacent insert segment are connected by an interrupted weld seam.

4. Gaiter according to claim 1, wherein the sleeve is evacuable, has a valve and the moldable mass is formed by loose particles.

5. Gaiter according to claim 1, wherein the moldable mass is formed by a dilatant fluid, optionally in admixture with particles.

6. Gaiter according to claim 1, wherein a shell segment movably arranged in the shell, which shell segment encompasses a terminal section of the insert at its second terminal section lying opposite to its first terminal section in which the retaining projections are arranged.

7. Gaiter according to claim 1, wherein an intermediate layer is arranged between the shell and the insert, which intermediate layer at least one recess arranged around a retaining projection.

8. Gaiter according to claim 1, wherein the retaining recesses extend from the concave inner side of the shell, wherein the opposite convex outer side of the shell has a closed surface.

9. Gaiter according to claim 1, wherein the insert is an elastic sleeve within which a gas-permeable inner layer filled with particles is arranged, which inner layer is subdivided into segments filled with particles.

10. Gaiter according to claim 1, wherein the insert is divided into interconnected insert segments, which are each segments of a sleeve, wherein the insert segments are interconnected by a conduit and have a common valve, or are separated from each other and each have a separate valve.

11. Gaiter according to claim 1, wherein the insert is divided into insert segments, at least one of which contains a dilatant fluid as a moldable mass.

12. Gaiter according to claim 6, wherein an insert segment is encompassed by a shell segment.

13. Gaiter according to claim 12, wherein the shell segment has at least one retaining projection which projects into a recess of the shell and movably guides the shell segment in the shell.

14. Gaiter according to claim 1, wherein in each case one shell with at least two inserts forms a system in which the inserts are interchangeable with one another and each insert has a different filling of particles and/or dilatant fluid as a moldable mass.

15. Gaiter according to claim 1, wherein the insert is surpassed by 1 to 15 mm at its circumferential edged by the shell encompassing it.
